(19) United States
(12) Patent Application Publication
(10) Pub. No.: US 2020/0121416 A1
(43) Pub. Date: Apr. 23, 2020

(54) MEDICAL INSTRUMENT HOLDER, ADAPTER FOR MEDICAL INSTRUMENT HOLDER, AND INSTRUMENT HOLDING DEVICE

(71) Applicant: OLYMPUS CORPORATION, Tokyo (JP)

(72) Inventors: Ryota SASAI, Tokyo (JP); Kazuo BANJU, Tokyo (JP)

(73) Assignee: OLYMPUS CORPORATION, Tokyo (JP)

(21) Appl. No.: 16/724,731

(22) Filed: Dec. 23, 2019

Related U.S. Application Data
(63) Continuation of application No. PCT/JP2017/024157, filed on Jun. 30, 2017.

(51) Int. Cl. A61B 90/50; F16M 11/08; F16M 13/02

(52) U.S. Cl. CPC A61B 90/50; A61B 1/00149; F16M 13/022; F16M 11/08

(57) ABSTRACT

A medical instrument holder for holding a medical instrument comprising: an adapter connected to a base portion via an arm; and an instrument holding portion configured to be capable of holding the medical instrument and connected to the adapter. The instrument holding portion includes: a positioning portion configured to perform positioning with respect to the adapter when mounted on the adapter; and a rod configured to be connected to the positioning portion and to be inserted into the adapter. The adapter includes: a contact portion to which an upper surface of the positioning portion contacts and which has an opening; a fitting hole configured to communicate with the opening of the contact portion and to extend in an insertion direction of the rod; and a window portion that is open to the fitting hole and is configured so that an insertion state of the rod can be visually recognized through the window portion when the positioning portion contacts to the contact portion.

### CROSS REFERENCE TO RELATED APPLICATIONS

This application is a continuation application based on a PCT Patent Application No. PCT/JP2017/024157, filed on Jun. 30, 2017, the content of which is incorporated herein by reference.

### BACKGROUND

### Technical Field

The present invention relates to a medical instrument holder for holding a medical instrument, an adapter for medical instrument holder, and an instrument holding device.

### Background Art

In a laparoscopic operation or the like, for the purpose of preventing hand shake caused by a scopist holding an endoscope such as a laparoscope or reducing fatigue of the scopist, a holder that holds the endoscope is used.

Unlike endoscopes that are sterilized before being used, holders cannot be sterilized in general, and therefore cannot be simply placed in an operating room that is a clean space. Usually, a holder is covered with a flexible drape formed of vinyl or the like when placed in an operating room.

When an endoscope is attached to a holder covered with a drape, the attaching operation becomes complicated. In relation to this, Japanese Unexamined Patent Application, First Publication No. 2005-253987 (hereinafter referred to as Patent Document 1) discloses a holder in which a sterilizable intermediate member is attached to the holder body and an endoscope is attached to the intermediate member.

### SUMMARY

In order to perform a stable operation during the operation, a medical instrument such as an endoscope needs to be securely attached to the intermediate member. In the holder of Patent Document 1, there may be a case in which it is difficult to determine whether the medical instrument is securely attached to the intermediate member.

The present invention is made in view of the situation described above, and an aim of the present invention is to provide a medical instrument holder which can confirm the connection state of a medical instrument and an intermediate member easily.

A medical instrument holder for holding a medical instrument comprising: an adapter connected to a base portion via an arm; and an instrument holding portion configured to be capable of holding the medical instrument and connected to the adapter. The instrument holding portion includes: a positioning portion configured to perform positioning with respect to the adapter when mounted on the adapter; and a rod configured to be connected to the positioning portion and to be inserted into the adapter. The adapter includes: a contact portion to which an upper surface of the positioning portion contacts and which has an opening; a fitting hole configured to communicate with the opening of the contact portion and to extend in an insertion direction of the rod; and a window portion that is open to the fitting hole and is configured so that an insertion state of the rod can be visually recognized through the window portion when the positioning portion contacts to the contact portion.

A plurality of window portions may be provided in a circumferential direction, and the rod may be provided with a marking extending in the circumferential direction, the marking being configured so that the insertion state can be visually recognized through the window portion when the positioning portion contacts to the contact portion.

The adapter may include: a base body configured to hold the instrument holding portion; and an operation ring attached to the base body so as to be relatively rotatable about an axis of the base body, the base body may have a support protrusion configured to hold the positioning portion, and the instrument holding portion may have an engaging surface configured to support the positioning portion by the support protrusion.

The base body may be formed in a substantially annular shape. An inner surface of the operation ring may have a short distance portion in which a distance between the inner surfaces facing each other is substantially the same as an outer diameter of the base body, and a long distance portion in which the distance between the inner surfaces facing each other may be larger than the outer diameter of the base body. When phases of the support protrusion and the short distance portion coincide with each other, the support protrusion may protrude to inside of the base body. When phases of the support protrusion and the long distance portion coincide with each other, the support protrusion may be capable of moving outward in a radial direction of the base body.

The operation ring may be connected to the base body via an elastic member. When the operation ring is released from a rotating state, state may be changed from a state in which phases of the support protrusion and the long distance portion coincide with each other to a state in which phases of the support protrusion and the short distance portion coincide with each other.

One of the contact portion and the positioning portion may have a plurality of positioning protrusions, and the other may have a concave portion into which the positioning protrusions can enter.

The instrument holding portion may include a slide lock arranged so as to sandwich a part of the adapter and a part of the instrument holding portion.

In a vertical direction of the adapter, positions of an upper end of the window portion and a top surface of the fitting hole may be substantially the same.

An adapter for medical instrument holder configured to connect a medical instrument to a base portion, wherein the adapter has: a contact portion to which an upper surface of a positioning portion that performs positioning with respect to the adapter when mounted on the adapter contacts, and which has an opening; a fitting hole configured to communicate with the opening of the contact portion and into which a rod connected to the positioning portion can be inserted; and a window portion that is open to the fitting hole and is configured so that an insertion state of the rod can be visually recognized through the window portion when the positioning portion contacts to the contact portion.

A plurality of window portions may be provided in a circumferential direction, and the rod may be provided with a marking extending in the circumferential direction, the marking being configured so that the insertion state can be visually recognized through the window portion when the positioning portion contacts to the contact portion.

The adapter may include: a base body configured to hold an instrument holding portion capable of holding the medical instrument and connected to the adapter; and an operation ring attached to the base body so as to be relatively rotatable about an axis of the base body, and the base body may have a support protrusion configured to hold the positioning portion.

The base body may be formed in a substantially annular shape. An inner surface of the operation ring may have a short distance portion in which a distance between the inner surfaces facing each other is substantially the same as an outer diameter of the base body, and a long distance portion in which the distance between the inner surfaces facing each other may be larger than the outer diameter of the base body. When phases of the support protrusion and the short distance portion coincide with each other, the support protrusion may protrude to inside of the base body. When phases of the support protrusion and the long distance portion coincide with each other, the support protrusion may be capable of moving outward in a radial direction of the base body.

The operation ring may be connected to the base body via an elastic member. When the operation ring is released from a rotating state, state may be changed from a state in which phases of the support protrusion and the long distance portion coincide with each other to a state in which phases of the support protrusion and the short distance portion coincide with each other.

One of the contact portion and the positioning portion may have a plurality of positioning protrusions, and the other may have a concave portion into which the positioning protrusions can enter.

The adapter may include a slide lock arranged so as to sandwich a part of the adapter and a part of an instrument holding portion capable of holding the medical instrument and connected to the adapter.

In a vertical direction of the adapter, positions of an upper end of the window portion and a top surface of the fitting hole may be substantially the same.

An instrument holding device used for attaching a medical instrument to a medical instrument holder via an adapter, wherein the instrument holding device includes: a rod that is inserted into a fitting hole that extends in communication with an opening provided in the adapter; and a positioning portion that is connected to the rod and contacts to a contact portion of the adapter to perform positioning with respect to the adapter, and the rod is configured so that an insertion state of the rod can be visually recognized through a window portion that is open to the fitting hole of the adapter when the positioning portion contacts to the contact portion.

The instrument holding device may include an engaging surface that can be supported by a support protrusion that is disposed on a base body configured to hold the instrument holding device.

The instrument holding portion may have a positioning portion that contacts to the contact portion when attached to the adapter, wherein one of the contact portion and the positioning portion provided on the base body may have a plurality of positioning protrusions, and the other may have a concave portion into which the positioning protrusions can enter.

The instrument holding device may include a slide lock arranged to sandwich a part of the adapter and a part of the instrument holding device.

### BRIEF DESCRIPTION OF THE DRAWINGS

FIG. 1 is an overall view of a medical instrument holder according to an embodiment of the present invention.

FIG. 2 is an enlarged view showing a distal end of an arm of the medical instrument holder.

FIG. 3 is a front view showing an adapter of the medical instrument holder.

FIG. 4 is a perspective view showing the adapter from below.

FIG. 5 is a bottom view of the adapter.

FIG. 6 is a view showing an instrument holding portion of the medical instrument holder to which the medical instrument is attached.

FIG. 7 is a partially enlarged view of the instrument holding portion.

FIG. 8 is a view schematically showing a concave shape of the instrument holding portion.

FIG. 9 is a view showing a state in which a holding member of the instrument holding portion is viewed from below.

FIG. 10 is a cross-sectional view showing a process of attaching the instrument holding portion to the adapter.

FIG. 11 is a cross-sectional view showing a state where a contact portion and a positioning portion are in contact with each other.

FIG. 12 is a cross-sectional view schematically showing an aspect of a contact between a positioning protrusion and a concave portion.

FIG. 13 is a diagram showing a state in which the instrument holding portion is completely attached to the adapter.

FIG. 14 is a diagram schematically showing an instrument holding portion and a medical instrument in a modified example of the present invention.

### DETAILED DESCRIPTION OF THE PREFERRED EMBODIMENTS

A medical instrument holder (which may be simply referred to hereinafter as a “holder”) according to an embodiment of the present invention will be described below with reference to the drawings.

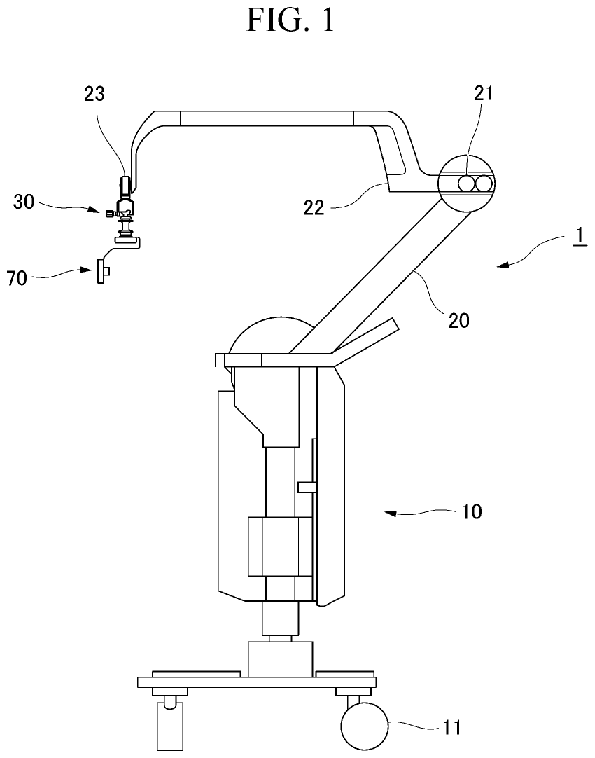

FIG. 1 is an overall view of a medical instrument holder 1 according to the present embodiment. The holder 1 includes a base portion 10, an arm 20 attached to the base portion 10, an adapter 30 attached to the distal end portion of the arm 20, and an instrument holding portion 70 (instrument holding device) attached to the adapter 30.

The base portion 10 includes casters 11 and is configured to be easily moved. However, the casters 11 are not essential and a stand type that does not include casters may be used.

The arm 20 is rotatably attached to the base portion 10. The arm 20 includes a plurality of joints 21, 22, and 23. By appropriately operating the joints 21, 22, and 23, the distal end portion of the arm 20 can be moved to a desired position. The number of joints provided in the arm 20, the aspect of moving, the arrangement, and the like can be set as appropriate.

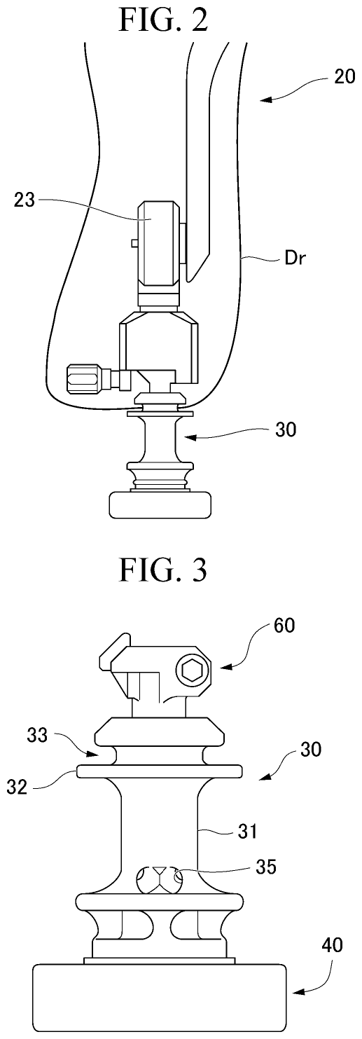

FIG. 2 is an enlarged view of the distal end portion of the arm 20. The adapter 30 is detachably attached to a portion further distal than the joint 23 provided on the most distal side in the arm 20. The joint 23 is also the most distal joint located on the most distal side in the holder 1.

FIG. 3 is a front view of the adapter 30. The adapter 30 includes a substantially cylindrical main body 31 (including a cylindrical shape; the same applies hereinafter), a mounting portion 40 provided at a lower portion of the main body 31, and a locking portion 60 provided at an upper portion of the main body 31.

A flange 32 that protrudes in the circumferential direction is provided on the upper side of the main body 31.

The groove-shaped portion between the upper end portion of the main body 31 and the flange 32 has a smaller outer diameter than the top and bottom, and functions as a drape attachment portion 33.

A fitting hole (described later) through which a part of the instrument holding portion 70 can enter is formed on the lower side of the main body 31. Parts of the outer peripheral surface of the main body 31 are removed in the vicinity of the upper end portion of the fitting hole. Thereby, window portions 35 communicating with the fitting hole 34 are formed in the main body 31. In the present embodiment, three window portions 35 are formed at equal intervals in the circumferential direction of the main body 31.

In the axial direction of the main body 31, the position of the upper end of each window portion 35 and the position of the upper end of the fitting hole 34 are set to be substantially the same.

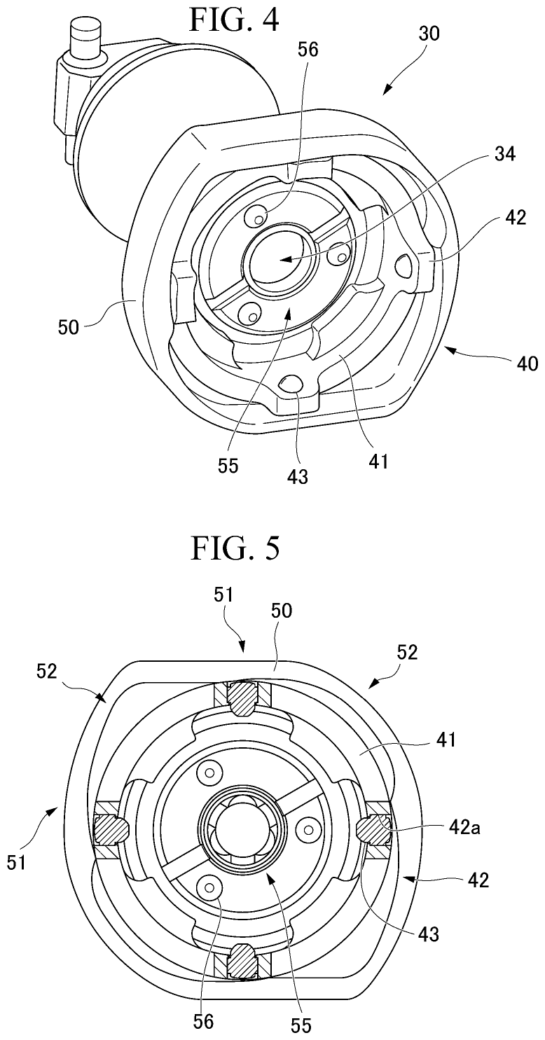

FIG. 4 is a perspective view showing the adapter 30 as viewed from below. FIG. 5 is a bottom view of the adapter 30 and shows a part of the mounting portion 40 in a cross section.

The mounting portion 40 includes a base body 41 that supports the mounted instrument holding portion 70, an operation ring 50 that is disposed so as to cover the outer peripheral surface of the base body 41, and a contact portion 55 that performs positioning of the mounted instrument holding portion 70.

The base body 41 is formed in a substantially annular shape and includes four support protrusions 42 protruding downward. The support protrusions 42 are arranged at equal intervals in the circumferential direction of the base body 41, and have through holes 42a that extend in the radial direction of the annular shape of the base body 41. In each through hole 42a, an engagement member 43 having a curved surface on a distal end side thereof is arranged with the distal end side facing the inside of the annular shape.

The operation ring 50 is attached to the base body 41 so as to be rotatable about the axis of the base body 41. When attached to the base body 41 as shown in FIG. 5, the inner surface of the operation ring 50 is formed to have two short distance portions 51 in which the distance between the inner surfaces facing each other is substantially the same as the outer diameter of the base body 41, and two long distance portions 52 in which the distance between the inner surfaces facing each other is larger than the outer diameter of the base body 41. The short distance portions 51 and the long distance portions 52 are formed so as to be alternately arranged at substantially equal intervals in the circumferential direction of the base body 41.

In the mounting portion 40 of the initial state shown in FIGS. 3 and 5, the phase of the support protrusion 42 on which the engaging member 43 is disposed and the phase of the short distance portion 51 of the operation ring 50 coincide. When the operation ring 50 is rotated 45 degrees around the axis of the base body 41, the phases of the support protrusion 42 and the long distance portion 52 coincide. The operation ring 50 is connected to the base body 41 through an elastic member such as a spring (not shown). When the operation ring 50 is released from a hand, the positional relationship between the operation ring 50 and the base body 41 returns to the initial state.

The central portion of the contact portion 55 is open and communicates with the fitting hole 34. The contact portion 55 has three positioning protrusions 56 that protrude downward. The positioning protrusions 56 have conical shapes with rounded tops, and are arranged at substantially equal intervals in the circumferential direction of the base body 41.

There is no restriction in particular on the shape of the locking portion 60, and as long as it can be detachably attached to the arm 20, a well-known appropriate shape and structure may be adopted.

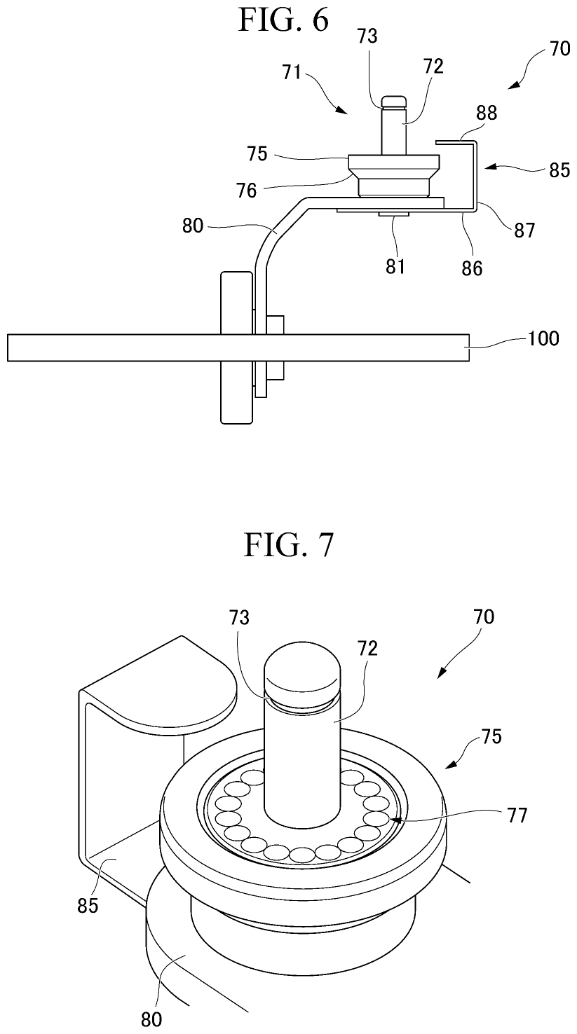

FIG. 6 is a diagram showing the instrument holding portion 70 to which the medical instrument 100 is attached. The instrument holding portion 70 includes a mounted portion mounting portion mounted on the adapter 30, a holding member 80 attached to the mounted portion, and a slide lock 85 attached to the holding member 80.

The mounted portion 71 includes a rod-shaped insertion portion (rod) 72 and a positioning portion 75 connected to the proximal end portion of the rod. The rod 72 is substantially cylindrical and has a dimension with which it is capable of entering the fitting hole 34, and a band-like marking 73 is provided at the distal end portion in the circumferential direction. The marking 73 can be formed by applying paint to the rod, providing a groove extending in the circumferential direction, or inserting an annular member of a predetermined color into the groove provided in the rod 72. Alternatively, only a desired shape such as a groove may be provided, and the marking may have the same color as other parts.

The marking 73 is formed at a position that can be viewed through the window portion 35 when the positioning portion 75 abuts the contact portion 55 (details will be described later).

The positioning portion 75 has a substantially cylindrical basic shape, and has a larger upper diameter from which the rod 72 protrudes. Thereby, on the outer peripheral surface of the positioning portion 75, an inclined engaging surface 76 extending toward its own axis line from the upper side to the lower side is formed.

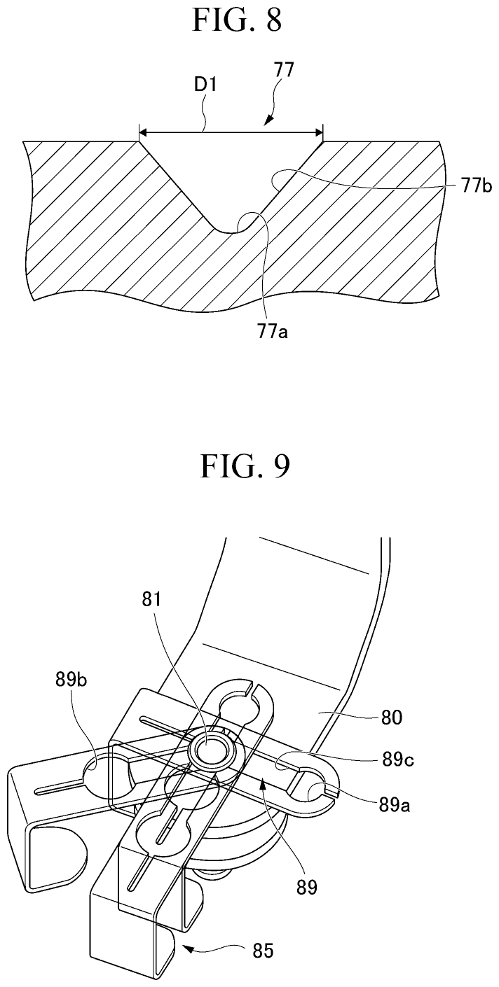

FIG. 7 is a partially enlarged view of the instrument holding portion 70. On the upper surface of the positioning portion 75, a plurality of bottomed concave portions 77 are formed side by side in the circumferential direction of the positioning portion 75. As schematically shown in FIG. 8, the inner surface of each concave portion 77 is configured by combining a spherical bottom surface 77a and a conical side surface 77b. The apex angle of the cone specified by the side surface 77b is set smaller than the apex angle of the conical shape of the positioning protrusion 56 formed on the contact portion 55. The diameter of the base part of the positioning protrusion 56 is larger than the diameter D1 of the opening circle at the top of the concave portion 77.

The holding member 80 is a substantially L-shaped member, and is fixed to the lower surface of the mounted portion 71. The holding member 80 has an insertion hole (not shown), and a part of the medical instrument 100 or the like can be fixedly inserted into the insertion hole.

The slide lock 85 is attached to a shaft 81 provided on the lower surface of the holding member 80. The slide lock 85 is a substantially J-shaped member including a sliding portion 86 attached to the holding member, a wall portion 87 extending perpendicularly to the sliding portion 86, and a pressing portion 88 extending parallel to each sliding portion 86 and perpendicular to the wall portion 87.

FIG. 9 is a diagram showing a state in which the holding member 80 is viewed from below. The sliding portion 86 is provided with a slit 89 for sliding the slide lock with respect to the holding member 80. The slit 89 has a first circular portion 89a at one end and a second circular portion 89b at the other end. In the slit 89, the diameter of the first circular portion 89a and the width of the linear portion 89c between the first circular portion 89a and the second circular portion 89b are smaller than the diameter of the end portion of the shaft 81. The diameter of the second circular portion 89b is larger than the diameter of the end portion of the shaft 81. Therefore, when the second circular portion 89b is moved to correspond to the position of the shaft 81, the slide lock 85 can be attached to and detached from the holding member 80. In a state where the portion other than the second circular portion 89b in the slit 89 is at the position of the shaft 81, the slide lock 85 can be rotated with respect to the holding member 80 around the shaft 81.

The operation at the time of use of the holder 1 of the present embodiment configured as described above will be described below.

Of the holder 1, the base portion 10 and the arm 20 are unclean areas because they cannot be sterilized due to their size, structure, or the like. Therefore, the base portion 10 and the arm 20 are entirely covered with a drape before use. The drape has an opening for attaching the adapter 30 to the arm 20.

Next, the user operates the locking portion 60 of the sterilized clean adapter 30 such that it enters the opening of the drape to be attached to the arm 20. Thereafter, the user fixes the edge of the opening of the drape Dr to the drape attachment portion 33 as shown in FIG. 2.

With these operations, the unsterilized unclean area of the holder 1 is covered with the drape Dr so as not to be exposed, and only a clean part of the adapter 30 protrudes from the opening of the drape Dr.

Next, the user attaches the instrument holding portion 70 to the adapter 30. The attachment of the instrument holding portion 70 may be performed either before or after the medical instrument 100 is attached to the instrument holding portion 70.

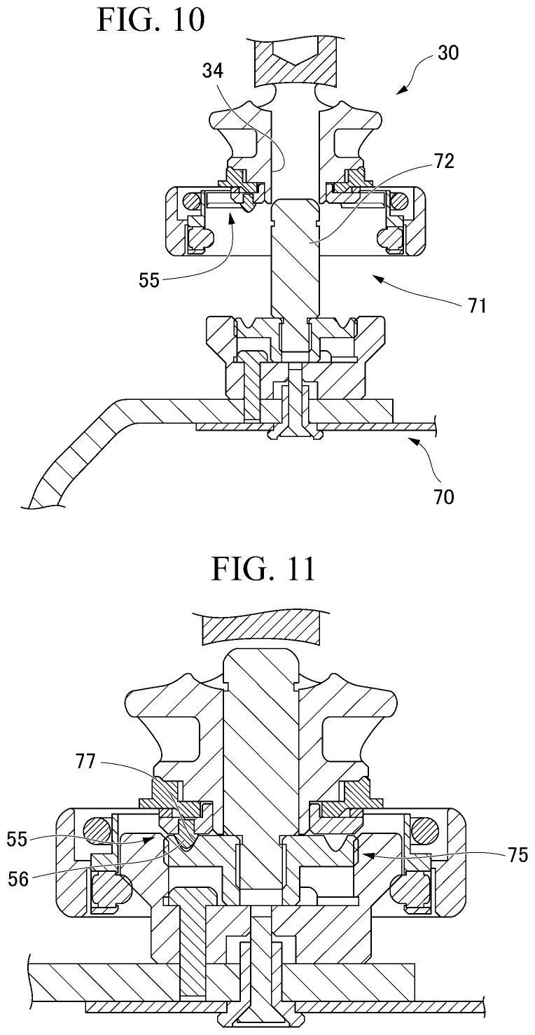

The user rotates the operation ring 50 to make the phases of the long distance portion 52 and the support protrusion 42 coincide with each other. As shown in FIG. 10, while holding the mounting portion 40 in the above-described state, the user operates the instrument holding portion 70 such that it approaches the adapter 30 from below with the mounted portion 71 facing upward, and causes the rod 72 to enter the fitting hole 34 from an opening of the contact portion 55.

As the rod 72 enters the fitting hole 34, the positioning portion 75 approaches the contact portion 55. The positioning portion 75 is in contact with the curved surface at the distal end of the engaging member 43 and moves upward from the engaging member 43 while pushing the engaging member 43 radially outward with respect to the adapter 30. At this time, since the phases of the long distance portion 52 and the support protrusion 42 coincide with each other, there is a gap between the support protrusion 42 and the inner surface of the operation ring 50, and a space for the engaging member 43 to move radially outward is reserved.

Subsequently, as shown in FIG. 11, the positioning protrusion 56 of the contact portion 55 enters the concave portion 77 of the positioning portion 75. Even when the phase of the positioning protrusion 56 is slightly shifted from the phase of the concave portion 77, the positioning portion 75 is slightly rotated with respect to the adapter 30 by being guided into the side surface 77b of the concave portion 77, and naturally, the phase of the positioning projection 56 and the concave portion 77 coincide with each other.

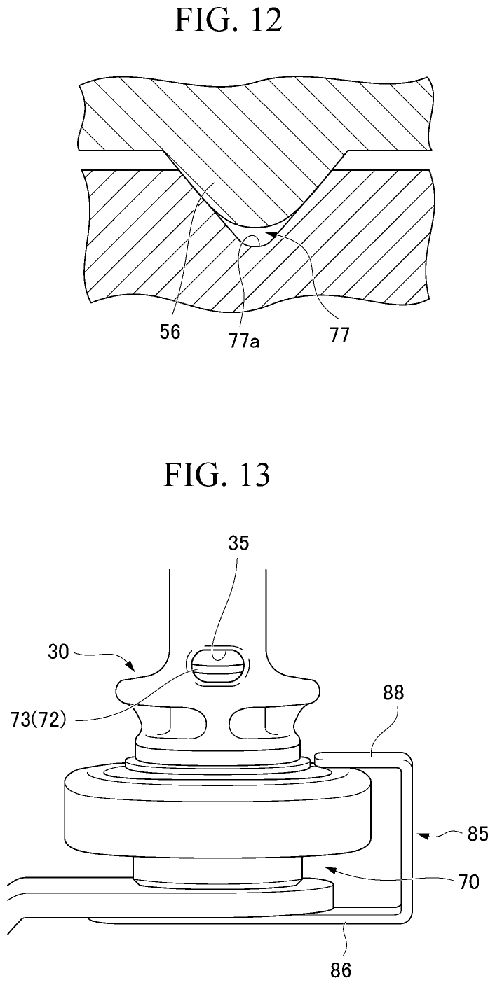

As described above, the apex angle of the conical shape of the concave portion 77 is smaller than the apex angle of the conical shape of the positioning protrusion 56, and the diameter of the base part of the positioning protrusion 56 is larger than the diameter of the opening circle above the concave portion 77. As a result, as shown in FIG. 12, the positioning protrusion 56 does not contact the bottom surface 77a of the concave portion 77 but contacts the opening edge of the concave portion 77 over the entire circumference.

With such an aspect of contact, after the positioning portion 75 abuts the contact portion 55, the positioning protrusion 56 does not move in the concave portion 77. Thereby, it is possible to prevent the mounted instrument holding portion 70 from rattling with respect to the adapter 30. By providing the three positioning protrusions 56, three such supporting points are ensured, and the effect of preventing backlash is further enhanced.

The user returns the operation ring 50 to the initial state while keeping the positioning protrusion 56 stable in the concave portion 77. Then, the phases of the short distance portion 51 and the support protrusion 42 coincide with each other so that there is almost no gap between the support protrusion 42 and the inner surface of the operation ring 50, and the engaging member 43 protrudes inside the base body 41. The protruding engaging member 43 comes into contact with the engaging surface 76 of the positioning portion 75, and the mounting portion 40 and the mounted portion 71 are engaged. While the operation ring 50 is in the initial state, the engagement member 43 cannot move radially outward with respect to the adapter 30, and thus the engagement state between the two is reliably maintained.

Finally, the user moves the slide lock 85 so that the sliding portion 86 and the pressing portion 88 sandwich a part of the adapter 30 and a part of the instrument holding portion 70 as shown in FIG. 13.

The mounting of the instrument holding portion 70 on the adapter 30 is thus completed.

In a state where the mounting portion 40 and the mounted portion 71 are engaged, the marking 73 of the rod 72 can be visually recognized from the window portion 35 as shown in FIG. 13. Although the user cannot directly confirm that the positioning protrusion 56 has sufficiently entered the concave portion 77, it is possible to confirm that the mounting portion 40 and the mounted portion 71 are securely engaged by visually checking the marking 73 from the window portion 35.

After mounting the instrument holding portion 70, the user performs a desired procedure using the medical instrument 100 attached to the instrument holding portion 70. The user can move and hold the medical instrument to a desired position by appropriately operating the arm 20.

Even if a failure occurs in the engagement between the mounting portion 40 and the mounted portion 71, it is possible to suitably prevent the instrument holding portion 70 from falling off of the adapter 30 by the slide lock 85, which is arranged so as to sandwich a part of the adapter 30 and a part of the instrument holding portion 70.

The user can finely adjust the positional relationship between the instrument holding portion 70 and the holder 1 by rotating the mounted instrument holding portion 70 with respect to the adapter 30. When the instrument holding portion 70 is rotated, each positioning protrusion 56, which has been fitted in the concave portions 77, goes over the opening edges of the concave portions 77 and comes out of the concave portions 77. When the instrument holding portion 70 is further rotated, each positioning protrusion 56 enters another concave portion 77 adjacent in the circumferential direction, and the positional relationship between the instrument holding portion 70 and the adapter 30 is maintained.

After the procedure is completed, the adapter 30 and the instrument holding portion 70 are cleaned and sterilized to be reused.

As for the adapter 30, the inside of the fitting hole 34 is cleaned. As described above, since the upper end surface (top surface) of the fitting hole 34 is substantially at the same height as the window portion 35, the hair of the cleaning brush that has entered from the window portion 35 can be sufficiently brought into contact with the top surface of the fitting hole 34, to perform cleaning without leaving an uncleaned area.

As for the instrument holding portion 70, the inside of each concave portion 77 is cleaned. As described above, since the bottom surface 77a of the concave portion 77 is spherical, there is no portion that the hair of the cleaning brush does not reach, and cleaning can be performed without leaving an uncleaned area. Furthermore, since the concave portion 77 is circular in a plan view, it can be suitably cleaned regardless of the direction from which the cleaning brush approaches.

Further, since the slide lock 85 can be rotated around the shaft 81 with respect to the holding member 80, the holding member 80 can be suitably cleaned while the slide lock 85 is rotated to change the portion of the holding member 80 covered by the slide lock 85. When the slide lock 85 is removed from the holding member 80, more careful cleaning can be performed.

As described above, since the holder 1 of the present embodiment is connected to the arm 20 and the instrument holding portion 70 via the adapter 30, even in a state where the unclean part of the holder 1 is completely covered with the drape, the medical instrument 100 can be easily attached.

In addition, since the most distal joint in the holder 1 is disposed on the arm 20, the adapter 30, which functions as an intermediate member, can have a simple structure without a joint. As a result, the adapter 30 can be easily cleaned and sterilized after use, and can be easily reused. As a result, sterilization is made unnecessary by completely covering all the joints of the holder 1 with the drape, and the usability of the holder 1 as a whole can be remarkably improved.

Further, with the above-described structure, the user can easily confirm that the instrument holding portion 70, which holds the medical instrument 100, is securely attached to the adapter 30 by checking the marking 73 from the window portion 35, and backlash of the instrument holding portion 70 after mounting is less likely to occur.

In addition, the adapter 30 and the instrument holding portion 70 can be easily cleaned without leaving an uncleaned area due to the structure described above. Therefore, it is easy to wash and sterilize it for reuse, and the maintenance cost of the holder can be reduced.

In the present embodiment, the slide lock 85 can be rotated around the shaft 81 relative to the holding member 80 and can be attached to and detached from the holding member 80. However, as long as one of the condition that the slide lock 85 can be relatively rotated around the shaft 81 and the condition that the slide lock 85 can be attached to and detached from the holding member 80 is satisfied, the instrument holding portion 70 can be cleaned sufficiently suitably.

Also, the slide lock may be attached to the adapter side.

In the present embodiment, the number of concave portions 77 and the intervals between them can be set as appropriate in consideration of the necessary fine adjustment resolution. Therefore, it is not essential in the present invention that the concave portions 77 be continuously provided in the circumferential direction, and the concave portions 77 may be formed discontinuously at appropriate intervals. Further, when fine adjustment is not required, the holder 1 can be configured so that the aspect of mounting of the instrument holding portion 70 is uniquely determined by making the number of concave portions and the number of positioning protrusions the same.

While preferred embodiments of the invention have been described and illustrated above, it should be understood that these are exemplary of the invention and are not to be considered as limiting. Additions, omissions, substitutions, and other modifications can be made without departing from the spirit or scope of the present invention.

For example, in the above-described embodiment, the example in which the mounting portion of the adapter includes the positioning protrusion and the mounted portion of the instrument holding portion includes the concave portion has been described. However, the concave portion may be provided on the side of the adapter, and the positioning protrusion may be provided on the side of the instrument holding portion.

In the above-described embodiment, an example in which the adapter has a fitting hole and the instrument holding portion has a rod has been described. However, a rod may be provided on the side of the adapter, and a fitting hole may be provided on the side of the instrument holding portion. The shape of the fitting hole and the rod can also be set as appropriate, and may be, for example, a prismatic shape.

Furthermore, the top surface of the fitting hole may be set slightly higher or lower than the upper end of the window portion. In this case, if there is a step between the upper end of the window portion and the top surface, cleaning becomes difficult, so it is preferable that the upper end of the window portion and the top surface be connected by a gentle slope shape or the like.

Furthermore, the holder of the present invention may include a known counter balance mechanism including a counter weight. At this time, a weight may be attached to the adapter or the instrument holding portion, and the holder may be configured so that the balance can be finely adjusted according to the medical instrument being held.

In the holder of the present invention, the number of window portions may be set as appropriate, but it is preferable to provide a plurality of window portions because the direction in which the marking can be visually recognized increases. A transparent member may be fitted into the window portion.

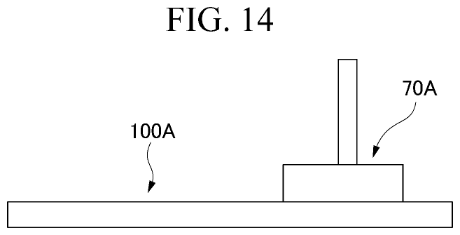

Furthermore, when the medical instrument is used exclusively for the holder of the present invention, as shown schematically in FIG. 14, the instrument holding portion 70A and the medical instrument 100A may be integrated. In this case, since the attachment of the medical instrument 100A to the holder is completed by attaching the instrument holding portion 70A to the adapter, the usability is further improved.

The medical instrument held by the holder of the present invention is not limited to an observation instrument such as an endoscope, but may be a high-frequency treatment tool, a treatment instrument such as a grasping forceps, or a needle holder.

The present invention can be applied to a medical instrument holder.

According to the medical instrument holder of the present invention, the connection state between the medical instrument and the intermediate member can be easily confirmed.

What is claimed is:

1. A medical instrument holder for holding a medical instrument comprising:
an adapter connected to a base portion via an arm; and
an instrument holding portion configured to be capable of holding the medical instrument and connected to the adapter,
wherein the instrument holding portion includes:
a positioning portion configured to perform positioning with respect to the adapter when mounted on the adapter; and
a rod configured to be connected to the positioning portion and to be inserted into the adapter,
the adapter includes:
a contact portion to which an upper surface of the positioning portion contacts and which has an opening;
a fitting hole configured to communicate with the opening of the contact portion and to extend in an insertion direction of the rod; and
a window portion that is open to the fitting hole and is configured so that an insertion state of the rod can be visually recognized through the window portion when the positioning portion contacts to the contact portion.

2. The medical instrument holder according to claim 1, wherein
a plurality of window portions are provided in a circumferential direction, and
the rod is provided with a marking extending in the circumferential direction, the marking being configured so that the insertion state can be visually recognized through the window portion when the positioning portion contacts to the contact portion.

3. The medical instrument holder according to claim 1, wherein
the adapter includes:
a base body configured to hold the instrument holding portion; and
an operation ring attached to the base body so as to be relatively rotatable about an axis of the base body,
the base body has a support protrusion configured to hold the positioning portion, and
the instrument holding portion has an engaging surface configured to support the positioning portion by the support protrusion.

4. The medical instrument holder according to claim 3, wherein
the base body is formed in a substantially annular shape,
an inner surface of the operation ring has a short distance portion in which a distance between the inner surfaces facing each other is substantially the same as an outer diameter of the base body, and a long distance portion in which the distance between the inner surfaces facing each other is larger than the outer diameter of the base body,
when phases of the support protrusion and the short distance portion coincide with each other, the support protrusion protrudes to inside of the base body, and,
when phases of the support protrusion and the long distance portion coincide with each other, the support protrusion is capable of moving outward in a radial direction of the base body.

5. The medical instrument holder according to claim 4, wherein
the operation ring is connected to the base body via an elastic member, and
when the operation ring is released from a rotating state, state is changed from a state in which phases of the support protrusion and the long distance portion coincide with each other to a state in which phases of the support protrusion and the short distance portion coincide with each other.

6. The medical instrument holder according to claim 1, wherein
one of the contact portion and the positioning portion has a plurality of positioning protrusions, and the other has a concave portion into which the positioning protrusions can enter.

7. The medical instrument holder according to claim 3, wherein
the instrument holding portion includes a slide lock arranged so as to sandwich a part of the adapter and a part of the instrument holding portion.

8. The medical instrument holder according to claim 1, wherein,
in a vertical direction of the adapter, positions of an upper end of the window portion and a top surface of the fitting hole are substantially the same.

9. An adapter for medical instrument holder configured to connect a medical instrument to a base portion, wherein
the adapter has:
a contact portion to which an upper surface of a positioning portion that performs positioning with respect to the adapter when mounted on the adapter contacts, and which has an opening;
a fitting hole configured to communicate with the opening of the contact portion and into which a rod connected to the positioning portion can be inserted; and
a window portion that is open to the fitting hole and is configured so that an insertion state of the rod can be visually recognized through the window portion when the positioning portion contacts to the contact portion.

10. The adapter for medical instrument holder according to claim 9, wherein
a plurality of window portions are provided in a circumferential direction, and
the rod is provided with a marking extending in the circumferential direction, the marking being configured so that the insertion state can be visually recognized through the window portion when the positioning portion contacts to the contact portion.

11. The adapter for medical instrument holder according to claim 9, wherein
the adapter includes:
a base body configured to hold an instrument holding portion capable of holding the medical instrument and connected to the adapter; and
an operation ring attached to the base body so as to be relatively rotatable about an axis of the base body, and
the base body has a support protrusion configured to hold the positioning portion.

12. The adapter for medical instrument holder according to claim 11, wherein
the base body is formed in a substantially annular shape,
an inner surface of the operation ring has a short distance portion in which a distance between the inner surfaces facing each other is substantially the same as an outer diameter of the base body, and a long distance portion in which the distance between the inner surfaces facing each other is larger than the outer diameter of the base body, and,
when phases of the support protrusion and the short distance portion coincide with each other, the support protrusion protrudes to inside of the base body, and,
when phases of the support protrusion and the long distance portion coincide with each other, the support protrusion is capable of moving outward in a radial direction of the base body.

13. The adapter for medical instrument holder according to claim 12, wherein
the operation ring is connected to the base body via an elastic member, and
when the operation ring is released from a rotating state, state is changed from a state in which phases of the support protrusion and the long distance portion coincide with each other to a state in which phases of the support protrusion and the short distance portion coincide with each other.

14. The adapter for medical instrument holder according to claim 9, wherein
one of the contact portion and the positioning portion has a plurality of positioning protrusions, and the other has a concave portion into which the positioning protrusions can enter.

15. The adapter for medical instrument holder according to claim 9, wherein
the adapter includes a slide lock arranged so as to sandwich a part of the adapter and a part of an instrument holding portion capable of holding the medical instrument and connected to the adapter.

16. The adapter for medical instrument holder according to claim 11, wherein,
in a vertical direction of the adapter, positions of an upper end of the window portion and a top surface of the fitting hole are substantially the same.

17. An instrument holding device used for attaching a medical instrument to a medical instrument holder via an adapter, wherein
the instrument holding device includes:
a rod that is inserted into a fitting hole that extends in communication with an opening provided in the adapter; and
a positioning portion that is connected to the rod and contacts to a contact portion of the adapter to perform positioning with respect to the adapter, and
the rod is configured so that an insertion state of the rod can be visually recognized through a window portion that is open to the fitting hole of the adapter when the positioning portion contacts to the contact portion.

18. The instrument holding device according to claim 17, comprising an engaging surface that can be supported by a support protrusion that is disposed on a base body configured to hold the instrument holding device.

19. The instrument holding device according to claim 17, comprising a positioning portion that contacts to the contact portion when attached to the adapter,
wherein one of the contact portion and the positioning portion provided on the base body has a plurality of positioning protrusions, and the other has a concave portion into which the positioning protrusions can enter.

20. The instrument holding device according to claim 17, comprising a slide lock arranged to sandwich a part of the adapter and a part of the instrument holding device.
